(19) United States
(12) Patent Application Publication
(10) Pub. No.: US 2005/0173304 A1
(43) Pub. Date: Aug. 11, 2005

(54) Method for producing hydrogen and apparatus for producing hydrogen

(76) Inventors: Hajime Kabashima, Saitama (JP); Jun Iwamoto, Saitama (JP)

Correspondence Address: ARENT FOX PLLC, 1050 CONNECTICUT AVENUE, N.W., SUITE 400, WASHINGTON, DC, 20036, US

(21) Appl. No.: 11/034,206

(22) Filed: Jan. 13, 2005

(30) Foreign Application Priority Data
Jan. 14, 2004 (JP) 2004-007199

(51) Int. Cl. C10G 11/08; C07C 27/06

(52) U.S. Cl. 208/115; 568/959

(57) ABSTRACT

A method for reforming a diesel fuel with high efficiency under a same condition as that of gasoline or the like to continuously produce hydrogen with high selectivity and high yield is provided. By allowing three catalysts having different functions from one another to be in a composite state and, then, controlling a reforming reaction of the diesel fuel, the diesel fuel is reformed with high efficiency under a same condition as that of gasoline or the like and, accordingly, hydrogen can continuously be produced with high efficiency and high yield.

### CROSS-REFERENCE TO RELATED APPLICATIONS

This application is based upon and claims the benefit of priority from the prior Japanese Patent Application No. 2004-007199 filed on Jan. 14 in 2004, the entire contents of which is incorporated herein by reference.

### FIELD OF THE INVENTION

The present invention relates to a method for producing hydrogen with high yield by reforming a diesel fuel with high efficiency.

### RELATED ART

Hydrogen energy is clean energy which has drawn attention as a future alternative energy for petroleum and, in recent years, has been utilized as, for example, an energy source for a fuel cell or an internal combustion engine. As far as the internal combustion engine is concerned, applications thereof for a hydrogen fuel engine and a hydrogen-enriched fuel engine and as a reducing agent for removing NOₓ have been studied. In recent years, as described above, various types of studies have been exerted on applications of hydrogen and, in line with the studies, various types of studies on methods for producing hydrogen have been progressed. As for the methods for producing hydrogen, a method for reforming a fuel has conventionally been studied and the fuel to be used for fuel reforming is mainly natural gas or gasoline as described in a Non-Patent Document 1: SEA Tech Pap Ser (Soc Automot Eng), [D0244B (0148-7191)], SAE-2001-01-0234, pg. 6, 2001.

On the other hand, although a diesel fuel is considered to be highly valuable in use as a hydrogen source, there are various types of problems therein. Specifically, for example, when the diesel fuel is reformed in the presence of an Rh—Pt-carried catalyst, firstly, such reaction requires a high temperature (from 800° C. to 1000° C.) is required for a reaction compared with reforming of gasoline. This is because, as a carbon value becomes larger, a reaction temperature necessary for the reforming becomes higher. Further, as the reaction temperature becomes higher, not only an energy lose becomes larger, but also the catalyst tends to be subjected to sintering. Secondly, a carbonaceous material tends to be deposited on the catalyst compared with the reforming of gasoline. This is because the diesel fuel comprises hydrocarbons having more carbon atoms than those of gasoline and, accordingly, it is difficult to perform the reforming. In order to suppress such deposition of the carbonaceous material, it is necessary to feed an excess amount of an oxidizing agent (steam, oxygen, or air) into a reactor and, when an excess amount of the steam is fed thereinto, a thermal efficiency is decreased and a larger amount of energy becomes necessary to obtain hydrogen, while, when an excess amount of oxygen is fed, hydrogen yield is decreased due to overburning as described in a Non-Patent Document 2: US DOE Report, ANL-CMT-CP-102382, May 9, 2000. Moreover, when the excess oxidizing agent is supplied, unreacted oxiding agent needs to be separated and recovered from the hydrogen.

### SUMMARY OF THE INVENTION

From the reasons as described above, a case in which a diesel fuel is used in production of hydrogen based on fuel reforming is conventionally scarcely found and it is a present situation that a method for producing hydrogen by reforming the diesel fuel has not been established. When hydrogen can efficiently be supplied from the diesel fuel through a continuous reforming reaction, it is considered that not only fuel for a fuel cell or an internal combustion engine can be diversified, but also such supply may contribute to an effective use of a hydrogen-containing resource and, accordingly, such establishment of the method for producing hydrogen by reforming the diesel fuel is extremely useful.

Therefore, an object of the invention is to provide a method for continuously producing hydrogen with high selectivity and high yield by reforming a diesel fuel with high efficiency under a same condition as that of gasoline or the like.

Then, the present inventors have exerted intensive studies for solving the aforementioned problems and, as a result, found that hydrogen can continuously be produced with high selectivity and high yield by reforming the diesel fuel with high efficiency under a same condition as that of gasoline or the like while controlling a reforming reaction of the diesel fuel by using a composite of three catalysts which have different functions from one another, to thereby achieve the invention. More specifically, according to the invention, following aspects are provided:

(1) a method for producing hydrogen, which produces hydrogen from a diesel fuel, comprising the steps of;

partially oxidizing a portion of the diesel fuel by using a first catalyst to generate hydrogen and heat;

cracking the other portion of the diesel fuel which has not been partially oxidized by using a second catalyst, the generated hydrogen and heat to generate hydrocarbons having smaller molecular weights than those of hydrocarbons contained in the diesel fuel; and

reforming the hydrocarbons having the smaller molecular weights by subjecting the hydrocarbons having the smaller molecular weights to a reforming reaction by using a third catalyst to generate hydrogen;

(2) the method for producing hydrogen as described in (1), in which the reforming reaction in the reforming step is performed by at least one technique selected from the group consisting of a steam reforming method, a partial oxidation method and an autothermal reforming method which is a combination of the steam reforming method and the partial oxidation method;

(3) the method for producing hydrogen as described in (1), in which the partially oxidizing step, the cracking step and the reforming step are performed in atmospheres of air, oxygen and steam;

(4) the method for producing hydrogen as described in (1), in which the partially oxidizing step, the cracking step and the reforming step are continuously performed;

(5) an apparatus for producing hydrogen, which produces hydrogen by using a diesel fuel, comprising:

a partially oxidation unit which partially oxidizes a portion of the diesel fuel by using a first catalyst to generate hydrogen and heat;

a cracking unit which cracks the other portion of the diesel fuel which has not been partially oxidized by using a second catalyst, the generated hydrogen and heat to generate hydrocarbons having smaller molecular weights than those of hydrocarbons contained in the diesel fuel; and

a reforming unit which subjects the hydrocarbons having smaller molecular weights to a reforming reaction by using a third catalyst to generate hydrogen;

(6) the apparatus for producing hydrogen as described in (5), in which the reforming reaction in the reforming step is performed by at least one technique selected from the group consisting of a steam reforming method, a partial oxidation method and an autothermal reforming method which is a combination of the steam reforming method and the partial oxidation method;

(7) the apparatus for producing hydrogen as described in (5) or (6), in which the partially oxidizing step, the cracking step and the reforming step are performed in atmospheres of air, oxygen and steam;

(8) the apparatus for producing hydrogen as described in any one of (5) to (7), in which the partially oxidizing step, the cracking step and the reforming step are continuously performed; and

(9) a method for producing gasoline, which produces gasoline from a diesel fuel, comprising the steps of:

partially oxidizing a portion of the diesel fuel by using a first catalyst to generate hydrogen and heat; and

cracking the other portion of the diesel fuel which has not been partially oxidized by using a second catalyst, the generated hydrogen and heat to generate hydrocarbons having smaller molecular weights than those of hydrocarbons contained in the diesel fuel.

A method for producing hydrogen according to the invention is schematically shown in FIG. 1. As is shown in FIG. 1, in the method for producing hydrogen according to the invention, after lightening of the diesel fuel, that is, conversion thereof into hydrocarbons which are easily reformed, is performed, hydrogen can continuously be produced with high selectivity and high yield by subjecting the converted hydrocarbons to reforming with high efficiency under a same condition as that of gasoline or the like. Therefore, the method for producing hydrogen according to the invention makes it possible to efficiently produce hydrogen by performing reforming of the diesel fuel which has conventionally been difficult. In more detail, as shown in FIG. 2, in the method for producing hydrogen according to the invention, a reforming catalyst is designed in view of lightening of the diesel fuel and a high efficient reforming of the lightened diesel fuel and is characterized in that hydrogen is produced via a partial oxidation to be performed by using a first catalyst, cracking to be performed by using a second catalyst and a reforming reaction to be performed by using a third catalyst. Namely, according to the invention, by allowing three catalysts having different functions from one another to be in a composite state and controlling the reforming reaction of the diesel fuel, the diesel fuel is reformed with high efficiency under the same condition as that of the gasoline or the like, to thereby produce hydrogen with high selectivity and high yield. Further, in the method for producing hydrogen according to the invention, a reaction is, as a whole, progressed in accordance with a chemical reaction formula (1) as described below, to thereby produce hydrogen. Still further, at the time of designing the reforming catalyst, it can be expected to realize a low temperature of the reforming reaction by increasing a degree of dispersion of carried metals or allowing them to be in a composite state and to lighten a carbonaceous material by adjusting acidity-alkalinity of a catalyst carrier. Furthermore, an acceleration of gasification and an acceleration of oxidation can be expected by adding, as a third component, at least one of an oxide of an alkali metal, such as potassium (K) or cesium (Cs), an oxide of an alkaline earth metal, such as calcium (Ca) or strontium (Sr) and an oxide of a rare earth metal, such as lanthanum (La).
CₙHₘ+½(n+1)H₂O+¼nO₂→½(2n−1)CO+½CO₂+½(n+m+1)H₂ Formula 1,
Partially Oxidizing Step

The method for producing hydrogen according to the invention comprises firstly a partially oxidizing step which partially oxidizes a portion of a diesel fuel that comprises hydrocarbons each having from about 8 to 23 carbon atoms by a first catalyst. An example of a partial oxidation reaction formula is shown below. Although a partial oxidation reaction is progressed in accordance with a chemical reaction formula (2) as shown below, hydrogen and heat which are generated in this partial oxidation reaction can be utilized in cracking in the next step. The term “partially oxidize a portion of a diesel fuel by a first catalyst” as used herein is intended to mean that a portion of hydrocarbon molecules each having from about 8 to 23 carbon atoms is partially oxidized and, accordingly, the rest of the hydrocarbon molecules is not partially oxidized. Further, as for the first catalyst, an ordinary partially-oxidizing catalyst can be used; for example, Rh/Al₂O₃ is favorably used.
C₈H₁₈+4O₂→8CO+9H₂ Formula 2,
Cracking Step

Next, the method for producing hydrogen according to the invention comprises a cracking step which performs cracking on the portion of the diesel fuel which has not been partially oxidized in the partially oxidizing step by using a second catalyst, and the hydrogen and heat which have been generated in the partially oxidizing step. In this cracking step, a reaction is progressed in accordance with a chemical reaction formula (3) as described below and, then, the diesel fuel is lightened while repeating a β-cleavage. The term “β-cleavage” as used herein is intended to mean that a carbenium ion is subjected to the β-cleavage by allowing a carbon-carbon bond at a β position of a carbon cation having 3 coordinations to be comparatively weakened and, since a generated first carbenium ion is unstable, it is isomerized into a second carbenium ion which is, then, subjected to the β-cleavage again. Therefore, by performing this cracking, the diesel fuel comprising hydrocarbons each having from about 8 to 23 carbon atoms is lightened to that comprising hydrocarbons each having from about 3 to 8 carbon atoms. Further, as for the second catalyst to be used for the cracking, an ordinary cracking catalyst can be used. For example, a mixture of Pt/SiO₂—Al₂O₃ and Pt/USY (Pt-containing ultra-stable Y-type zeolite) is favorably used.
RCH₂—C⁺H—CH₂—CH₂R′→RCH₂—CH═CH₂+C⁺H₂R′ Formula 3,
Reforming Step

The method for producing hydrogen according to the invention comprises a reforming step which produces hydrogen by allowing the diesel fuel which has been lightened by cracking to perform a reforming reaction by a third catalyst. By lightening the diesel fuel by the cracking step, it becomes possible to perform an efficient reforming even at a low temperature. Further, as for the third catalyst, an ordinary reforming catalyst can be used. For example, Rh—Pt/Al₂O₃ is favorably be used.

The reforming reaction in the reforming step can be performed by at least one technique of a steam reforming mthod, a partial oxidation method and an autothermal reforming method which is a mixture of these methods. On this occasion, the term “steam reforming method” as used herein is intended to mean a method in which hydrocarbons are reformed by steam to generate hydrogen, carbon monoxide and carbon dioxide. Further, the term “partial oxidation method” is intended to mean, as described above, a method in which hydrocarbons are reformed by using oxygen in the air, a high-purity oxygen or the like to generate hydrogen and carbon monoxide. In the method for producing hydrogen according to the invention, hydrogen can be produced by adopting at least one of these methods as the reforming reaction.

Further, the method for producing hydrogen according to the invention is characterized in that the partially oxidizing step, the cracking step and the reforming step are performed in a same reactor under atmospheres of air, oxygen and steam to produce hydrogen.

Further, the method for producing hydrogen according to the invention can be performed by any one of a batch system and a continuous system. When a reaction gas is continuously fed into a reactor, the reforming reaction using the catalyst can stably be performed while least decreasing hydrogen yield and, therefore, the continuous system is preferred.

As has been described above, by performing the method for producing hydrogen according to the invention, the diesel fuel can be reformed with high efficiency under a same condition as that of gasoline or the like, to thereby continuously produce hydrogen with high selectivity and high yield. Further, reforming of the diesel fuel can be performed at a low temperature, to thereby reduce an amount of an oxidizing agent such as water or air to be used. In addition to these features, various types of effects as described below can be attained. Firstly, such problems as sintering of the catalyst and coking on the catalyst can be reduced and, then, deterioration of activity of the catalyst is suppressed and, accordingly, an extension of a service life of the catalyst can be attained. For this reason, an amount of the catalyst to be used can be suppressed and, accordingly, a realization of a low cost can be expected. Further, since coking on the catalyst can be suppressed, a pressure loss of the catalyst can be suppressed and, accordingly, a liquid hourly space velocity (LHSV) can be increased. Secondly, a general-purpose material can be used in a reaction apparatus and, accordingly, a fabrication cost of the reactor can be reduced. Still further, it becomes possible to design a compact reaction apparatus. Thirdly, since reforming is performed after lightening of the diesel fuel is performed, a molar ratio among hydrogen, carbon monoxide and carbon dioxide can be controlled. Fourthly, not only a start-up time of the reforming reaction can be shortened, but also energy to be consumed at the start-up time of the reforming reaction can be small compared with a conventional method.

Further, same effects as described above can also be obtained by using the apparatus for producing hydrogen according to the invention. Further, even when the reaction is forced to be terminated at the cracking step without further proceeding to the reforming step, gasoline instead of hydrogen can be obtained.

According to the invention, the diesel fuel is reformed with high efficiency under a same condition as that of gasoline or the like and, accordingly, hydrogen can continuously be produced with high selectivity and high yield.

### BRIEF DESCRIPTION OF THE DRAWINGS

FIG. 1 is a view for explaining a mechanism of a method for producing hydrogen according to the invention;

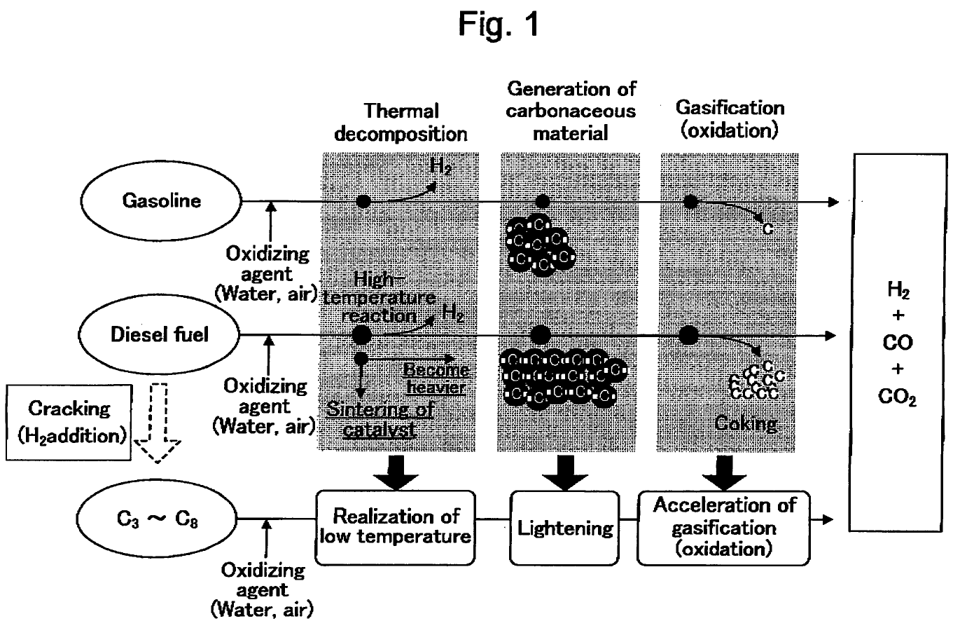

FIG. 2 is a view for explaining a mechanism of a method for producing hydrogen according to the invention;

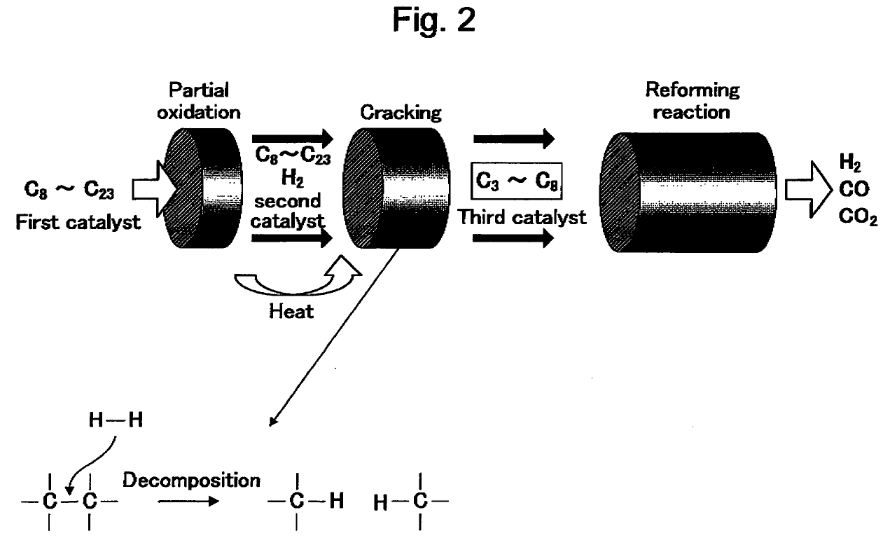

FIG. 3 is a diagram schematically showing an apparatus 10 for producing hydrogen according to the invention; and

FIG. 4 is a view for explaining a method for producing hydrogen according to a present embodiment.

### DESCRIPTION OF THE PREFERRED EMBODIMENT

Hereinafter, embodiments according to the invention will be described with reference to the accompanying drawings.

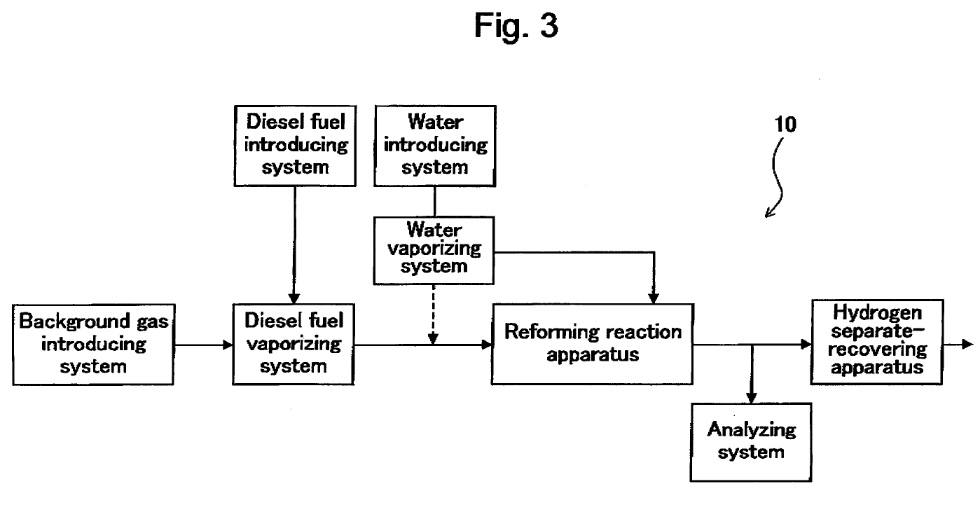

FIG. 3 is a diagram showing a schematic constitution of an apparatus 10 for producing hydrogen according to the invention. As shown in FIG. 3, the apparatus 10 for producing hydrogen according to the invention, which is a continuously-producing-type apparatus for producing hydrogen, comprises a diesel fuel introducing system, a water introducing system, a background gas introducing system, a diesel fuel vaporizing system, a water vaporizing system, a reforming reaction apparatus, a hydrogen separate-recovering apparatus and an analyzing system. A diesel fuel is mixed with a background gas and, then, the resultant mixed gas is introduced into a gas flow rate controlling system provided with a flow meter passing through a stop valve, a float control valve and the like. The introduced mixed gas is, then, introduced into the reforming reaction apparatus and reformed therein, to thereby generate a hydrogen-containing gas. Components of the generated hydrogen-containing gas are, then, analyzed by the analyzing system such as a gas chromatography and, thereafter, the hydrogen-containing gas is sent to the hydrogen separate-recovering apparatus and, subsequently, hydrogen is recovered therein. A waste gas remained after removing hydrogen is treated by a waste gas treating system. Further, in the method for producing hydrogen according to the invention, besides hydrogen, carbon monoxide, carbon dioxide and hydrocarbons are generated as by-products.

A raw material to be used in the method for producing hydrogen according to the invention is the diesel fuel. The diesel fuel, which comprises hydrocarbons each having, mainly, from 8 to 23 carbon atoms, contains alkanes, alkenes, aromatic compounds and the like. As for reforming agents, at least one article selected from among water, air, oxygen and carbon dioxide can be used. As for water, not only purified water, but also rain water, tap water, primary treated waste water and the like can be used. Further, as described above, as for the three catalysts which are used in the method for producing hydrogen according to the invention, namely, a partially oxidizing catalyst denoted as a first catalyst, a cracking catalyst denoted as a second catalyst and a reforming catalyst denoted as a third catalyst, respective conventionally ordinary ones can be used. The apparatus for producing hydrogen is not particularly limited and any of conventionally known ones can be used. As for such apparatuses for producing hydrogen, a fixed bed flow-type reactor, a batch-type reactor and the like are mentioned. The reforming reaction can be performed in the temperature range of from 500° C. to 900° C. and a concentration of hydrogen can be adjusted by a vapor pressure of products generated by the reforming.

According to the invention, although the reforming reaction may be performed by directly introducing the diesel fuel into the reforming reaction apparatus, it is preferable that, after the diesel fuel is vaporized in a background gas such as the air, the resultant gas mixture is introduced into the reforming reaction apparatus. Further, when the air is used as the background gas, a weight ratio of air/fuel is preferably in the range of from 2 to 20. This is because, as a concentration of oxygen in the background gas becomes higher, hydrogen generated in the reforming reaction is oxidized and more converted into water to reduce a recovery rate of a hydrogen gas.

Further, in order to perform the reforming reaction according to the invention, it is preferable that the diesel fuel is mixed with the background gas in advance to prepare a reaction gas and, then, the prepared reaction gas is introduced into the reforming reaction apparatus. A concentration of the reaction gas and a gas flow rate thereof give a large influence to hydrogen yield. In order to maximize a quantity of hydrogen to be produced per hour, it is preferable that a concentration of the diesel fuel is increased to allow the gas flow rate to be large. Ordinarily, the diesel fuel and the background gas are mixed with each other such that the ratio of air/fuel becomes from 4 to 10. The reaction pressure is not particularly limited and is preferably one atm. In an atmosphere of a normal pressure.

Hereinafter, embodiments according to the invention will be described, but it is to be understood that the invention is not limited thereto.

Preparation of Catalyst

First Catalyst (Partially Oxidizing Catalyst): Rh/Al₂O₃

At the time of preparing a partially oxidizing catalyst which is a first catalyst, firstly, γ-Al₂O₃ was prepared. Specifically, about 100 g of γ-Al₂O₃ which had been prepared in advance was dispersed in 200 mL of ultra-pure water contained in a 500-mL beaker. The resultant dispersion was added with a stirrer piece made of TEFLON® and, then, mildly stirred by a magnetic stirrer equipped with a hot plate for several minutes at room temperature. After water was removed therefrom, such operation was repeated three times. The treated γ-Al₂O₃ was covered such that a dust did not enter and, then, subjected to a vacuum-drying overnight at 80° C., to thereby remove moisture by evaporation therefrom. After such drying treatment was terminated, the resultant γ-Al₂O₃ was transferred to a storage container which was, then, stored in a desiccator until it was used.

Next, the partially oxidizing catalyst Rh/Al₂O₃, which is the first catalyst, was prepared by a precipitation-sedimentation method. 25 g of dry powder of Al₂O₃ was weighed out and, separately, rhodium nitrate: Rh(NO₃)₃ was weighed out to be 5% by weight in terms of a rhodium metal. Then, γ-Al₂O₃, 350 mL of ultra-pure water and an organic alkali: N(CH₃)₄OH.5H₂O were added to a 500-mL three-necked flask and, then, mixed while being heated, to thereby prepare a slurry having a temperature of 60° C. An aqueous solution of rhodium nitrate was slowly added to the 500-mL three-necked flask in a small amount at a time (in a slowly-adding manner such that the temperature of 60° C. was held). After such addition of the aqueous solution of rhodium nitrate was completed, the resultant mixture was stirred for one hour at 60° C. and, then, the resultant slurry was, after water contained therein was removed by evaporation, put in an oven at 100° C. and dry-solidified therein in 12 hours. The resultant dry-solidified catalyst was put in an electric oven at 600° C. and calcined therein for 4 hours.

Second Catalyst (Cracking Catalyst): Pt/SiO₂—Al₂O₃, Pt/USY

At the time of preparing a cracking catalyst which is a second catalyst, firstly, SiO₂—Al₂O₃ was prepared. About 100 g of SiO₂—Al₂O₃ which had been prepared in advance was dispersed in 200 mL of ultra-pure water contained in a 500-mL beaker. The resultant dispersion was added with a stirrer piece made of TEFLON® and, then, mildly stirred by a magnetic stirrer equipped with a hot plate for several minutes at room temperature. After water was removed therefrom, such operation was repeated three times. The treated SiO₂—Al₂O₃ was covered such that a dust did not enter and, then, subjected to a vacuum-drying overnight at 80° C., to thereby remove moisture by evaporation therefrom. After such drying treatment was terminated, the resultant SiO₂—Al₂O₃ was transferred to a storage container which was, then, stored in a desiccator until it was used.

Besides, USY was also prepared. About 100 g of ultra-stable Y-type zeolite (USY) which had been prepared in advance was dispersed in 200 mL of ultra-pure water contained in a 500-mL beaker. The resultant dispersion was added with a stirrer piece made of TEFLON® and, then, mildly stirred by a magnetic stirrer equipped with a hot plate for several minutes at room temperature. After water was removed therefrom, such operation was repeated three times. The treated USY was covered such that a dust did not enter and, then, subjected to a vacuum-drying overnight at 80° C., to thereby remove moisture by evaporation therefrom. After such drying treatment was terminated, the resultant USY was transferred to a storage container which was, then, stored in a desiccator until it was used.

Next, Pt/SiO₂—Al₂O₃ was prepared by an impregnation method. 25 g of dry powder of SiO₂—Al₂O₃ was weighed out and, separately, hydrogen chloroplatinate: H₃PtCl₆.5.3H₂O was weighed out to be 1% by weight in terms of a platinum metal. Then, hydrogen chloroplatinate was dissolved in 200 mL of ultra-pure water contained in a 500-mL beaker and, after a stirrer piece made of TEFLON® was added into the resultant aqueous solution of platinum, the aqueous solution of platinum was stirred by a magnetic stirrer such that platinum in the aqueous solution was allowed to be in a uniformly dispersed state. SiO₂—Al₂O₃, which had been weighed out accurately, was slowly added to the aqueous solution of platinum in a small amount at a time. The resultant dispersion containing SiO₂—Al₂O₃ was slowly stirred by a magnetic stirrer for 60 minutes and, then, left to stand still for 6 hours. While being heated by a hot plate and being stirred by a glass stick, the dispersion in a slurry state was dry-solidified by evaporation and, then, the dry-solidified article was put in an oven at 100° C. for 12 hours. The resultant dry-solidified catalyst was put in an electric oven at 500° C. and calcined therein for 4 hours.

Further, similarly, Pt/USY was prepared by an impregnation method. 25 g of dry powder of USY was weighed out and, separately, hydrogen chloroplatinate: H₃PtCl₆.5.3H₂O was weighed out to be 1% by weight in terms of a platinum metal. Then, hydrogen chloroplatinate was dissolved in 200 mL of ultra-pure water contained in a 500-mL beaker and, after a stirrer piece made of TEFLON® was added into the resultant aqueous solution of platinum, the aqueous solution of platinum was stirred by a magnetic stirrer such that platinum therein was allowed to be in a uniformly dispersed state. USY, which had been weighed out accurately, was slowly added to the aqueous solution of platinum in a small amount at a time. The resultant dispersion containing USY was slowly stirred by a magnetic stirrer for 60 minutes and, then, left to stand still for 6 hours. While being heated by a hot plate and being stirred by a glass stick, the dispersion in a slurry state was dry-solidified by evaporation and, then, the dry-solidified article was put in an oven at 100° C. for 12 hours. The resultant dry-solidified catalyst was put in an electric oven at 400° C. and calcined therein for 4 hours.

Lastly, 10 g each of the prepared Pt/SiO₂—Al₂O₃ and Pt/USY was weighed out and, then, these catalysts in powder form are physically mixed with each other while being crushed in an agate mortar, to thereby prepare a mixed catalyst of Pt/SiO₂—Al₂O₃ and Pt/USY.

Third Catalyst (Reforming Catalyst): Rh—Pt/Al₂O₃

A reforming catalyst: Rh—Pt/Al₂O₃, which is a third catalyst, was prepared by an impregnation method. Specifically, 20 g of powder of Rh/Al₂O₃, which had been prepared as the partially oxidizing catalyst as described above, was weighed out and, separately, hydrogen chloroplatinate: H₃PtCl₆.5.3H₂O was weighed out to be 1% by weight in terms of a platinum metal. Then, hydrogen chloroplatinate was dissolved in 200 mL of ultra-pure water contained in a 500-mL beaker and, after a stirrer piece made of TEFLON® was added into the resultant aqueous solution of platinum, the aqueous solution of platinum was stirred by a magnetic stirrer such that platinum therein was allowed to be in a uniformly dispersed state. Rh/Al₂O₃, which had been weighed out accurately, was slowly added to the aqueous solution of platinum in a small amount at a time. The resultant dispersion containing Rh/Al₂O₃ was slowly stirred by a magnetic stirrer for 60 minutes and, then, left to stand still for 6 hours. While being heated by a hot plate and being stirred by a glass stick, the dispersion in a slurry state was dry-solidified by evaporation and, then, the dry-solidified article was put in an oven at 100° C. for 12 hours. The resultant dry-solidified catalyst was put in an electric oven at 500° C. and calcined therein for 4 hours.

Reforming Diesel Fuel

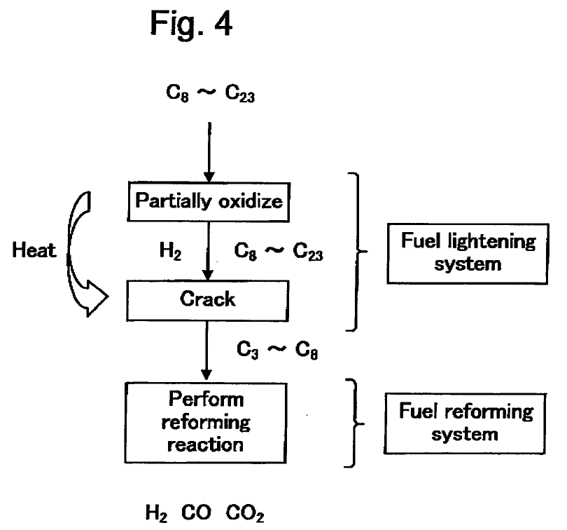

The diesel fuel was treated by using Rh/Al₂O₃ as the partially oxidizing catalyst, a mixture of Pt/SiO₂—Al₂O₃ and Pt/USY (Pt-containing ultra-stable Y-type zeolite) as the cracking catalyst and Rh—Pt/Al₂O₃ as the reforming catalyst, as described above, and, also, by making use of the apparatus 10 for producing hydrogen as shown in FIG. 3. The catalyst used in the reforming reaction apparatus and an outline of the reforming reaction are shown in FIG. 4. The reforming reaction was performed under conditions that a liquid hourly space velocity (LHSV) against the catalyst was in the range of from 0.5 to 20; a steam (mol)/carbon (mol) ratio, which is a ratio of steam against the diesel fuel, was in the range of from 0 to 5; and a reaction temperature was in the range of from 500° C. to 1000° C. Further, at the time of analyzing a generated gas, such analysis was performed by using a GC (trade name: GC-390B, Unipack S; available from GL Science) equipped with a hydrogen flame ionization detector (FID) as far as organic compounds are concerned and a GC (trade name: GC-390B, MS-5A; available from Shimadzu) equipped with a thermal conductivity detector (TCD) as far as H₂ is concerned.

Yield of hydrogen obtained as a result of reforming the diesel fuel by using the apparatus 10 for producing hydrogen according to the invention is shown in Table 1. In either of a case of the conventionally ordinary technique in which the catalyst is singly used and another case of the method for producing hydrogen according to the invention in which three catalysts, which are allowed to be in a composite state, are used, hydrogen yield was more increased as the reaction temperature was raised higher. At the time of the LHSV being 1 and the steam/carbon ratio being 1, in a conventionally ordinary technique, a low hydrogen yield was only obtained at a reaction temperature of 623° C. or less. Further, even in a case in which the reaction temperature was raised, for example, upto 923° C., the hydrogen yield was only 63.4%. To contrast, by the method for producing hydrogen according to the invention, even when the reaction temperature was 623° C., the hydrogen yield was 67.2%, which was about 7 times the hydrogen yield obtained by the conventionally ordinary technique. Further, when the reaction temperature was raised upto 723° C., the hydrogen yield was increased to 88.5%. From these results, it has been confirmed that, by the method for producing hydrogen according to the invention, namely, by allowing three catalysts having different functions from one another against reforming of the diesel fuel to be in a composite state and, also, by controlling the reforming reaction, hydrogen can be produced with high efficiency at a lower temperature than that of the conventional method.

TABLE 1

| | | Reaction | |
|---|---|---|---|
| | | temperature | Hydrogen |
| | Catalyst | (° C.) | yield (%) |
| Ordinary | Rh—Pt/Al₂O₃ | 523 | 4.8 |
| technique | | 623 | 9.9 |
| | | 723 | 49.8 |
| | | 773 | 60.5 |
| | | 923 | 63.4 |
| Present technique | Rh/Al₂O₃ + Pt/ | 523 | 27.3 |
| | USY | 623 | 67.2 |
| | Pt/SiO₂—Al₂O₃ + Rh—Pt/ | 723 | 88.5 |
| | Al₂O₃ | | |

LHSV = 1, Steam/Carbon = 1

Further, when the reaction temperature was set to be 623° C. under conditions that the LHSV is 1 and the steam/carbon ratio is 1 using the apparatus for producing hydrogen according to the invention, a hydrogen yield of from 66.3% to 70.8% was maintained for 10 hours in a row. In view of these results, it has been confirmed that the method for producing hydrogen according to the invention can be performed for long hours in a continuously stable manner.

1. A method for producing hydrogen, which produces hydrogen from a diesel fuel, comprising the steps of:
partially oxidizing a portion of the diesel fuel by using a first catalyst to generate hydrogen and heat;
cracking the other portion of the diesel fuel which has not been partially oxidized by using a second catalyst, the generated hydrogen and heat to generate hydrocarbons having smaller molecular weights than those of hydrocarbons contained in the diesel fuel; and
reforming the hydrocarbons having the smaller molecular weights by subjecting the hydrocarbons having the smaller molecular weights to a reforming reaction by using a third catalyst to generate hydrogen.

2. The method for producing hydrogen according to claim 1, wherein the reforming reaction in the reforming step is performed by at least one technique selected from the group consisting of a steam reforming method, a partial oxidation method and an autothermal reforming method which is a combination of the steam reforming method and the partial oxidation method.

3. The method for producing hydrogen according to claim 1, wherein the partially oxidizing step, the cracking step and the reforming step are performed in atmospheres of air, oxygen and steam.

4. The method for producing hydrogen according to claim 2, wherein the partially oxidizing step, the cracking step and the reforming step are performed in atmospheres of air, oxygen and steam.

5. The method for producing hydrogen according to claim 1, wherein the partially oxidizing step, the cracking step and the reforming step are continuously performed.

6. The method for producing hydrogen according to claim 2, wherein the partially oxidizing step, the cracking step and the reforming step are continuously performed.

7. The method for producing hydrogen according to claim 3, wherein the partially oxidizing step, the cracking stop and the reforming step are continuously performed.

8. The method for producing hydrogen according to claim 4, wherein the partially oxidizing step, the cracking step and the reforming step are continuously performed.

9. An apparatus for producing hydrogen, which produces hydrogen by using a diesel fuel, comprising:
a partially oxidation unit which partially oxidizes a portion of the diesel fuel by using a first catalyst to generate hydrogen and heat;
a cracking unit which cracks the other portion of the diesel fuel which has not been partially oxidized by using a second catalyst, the generated hydrogen and heat to generate hydrocarbons having smaller molecular weights than those of hydrocarbons contained in the diesel fuel; and
a reforming unit which subjects the hydrocarbons having smaller molecular weights to a reforming reaction by using a third catalyst to generate hydrogen.

10. A method for producing gasoline, which produces gasoline from a diesel fuel, comprising the steps of:
partially oxidizing a portion of the diesel fuel by using a first catalyst to generate hydrogen and heat; and
cracking the other portion of the diesel fuel which has not been partially oxidized by using a second catalyst, the generated hydrogen and heat to generate hydrocarbons having smaller molecular weights than those of hydrocarbons contained in the diesel fuel.
